(19) United States
(12) Patent Application Publication
(10) Pub. No.: US 2007/0264716 A1
(43) Pub. Date: Nov. 15, 2007

(54) Methods and materials for the transformation of the yeast Pichia ciferrii

(75) Inventors: Daniel Boergel, Frankfurt am Main (DE); Thomas Hueller, Marl (DE); Steffen Schaffer, Herten (DE)

Correspondence Address: OLIFF & BERRIDGE, PLC, P.O. BOX 19928, ALEXANDRIA, VA, 22320, US

(73) Assignee: COSMOFERM B.V., DELFT (NL)

(21) Appl. No.: 11/797,745

(22) Filed: May 7, 2007

(30) Foreign Application Priority Data
May 10, 2006 (EP) 06113772.5

(51) Int. Cl. C12N 15/74

(52) U.S. Cl. 435/483; 435/254.23

(57) ABSTRACT

The present invention discloses a transformation system useful for the genetic engineering of the industrial yeast Pichia ciferrii. It describes the isolation of auxotrophic mutants of this yeast and nucleotide sequences able to complement the auxotrophic requirements of these strains. The transformation system is based on mutant Pichia ciferrii cells auxotrophic for uracil or lysine, which are transformed with plasmids containing the URA3 or LYS2 genes as selection markers. Novel URA3 and LYS2 genes are also disclosed.

The industrial yeast Pichia ciferrii is known for its unique ability to produce high levels of sphingoid bases and derivatives thereof, mainly C18-phytosphingosine and acetylated derivatives thereof (Wickerham and Stodola, Formation of extracellular sphingolipids by microorganisms. I. Tetraacetylphytosphingosine from Hansenula ciferri. J Bacteriol. 1960 October;80:484-91). These can be extracted and chemically converted into corresponding ceramides (see e.g. WO 93/20038). Both sphingoid bases and ceramides are specialty chemicals of considerable commercial significance for food, pharmaceutical and cosmetic applications.

Consequently, it would be desirable to design Pichia ciferrii strains capable of efficiently producing sphingolipids other than phytosphingosine, e.g. sphingosine. However, sphingolipids are produced via a complex biosynthetic pathway, which is composed of multiple enzymatic steps required for the biosynthesis of a given sphingolipid, but also contains several branching points potentially being detrimental for the production of a given sphingolipid. Therefore, engineering the sphingolipid biosynthetic pathway, but also other biosynthetic pathways, in Pichia ciferrii will require a multitude of rational modifications of the genetic information naturally contained in this industrial yeast. The rational modification of genetic material requires the availability of suitable transformation systems. However, only one such system has so far been described for Pichia ciferrii (Bae et al., Integrative transformation system for the metabolic engineering of the sphingoid base-producing yeast Pichia ciferrii. Appl Environ Microbiol. 2003. 69:812-9; WO 99/57279). It relies on a modified ribosomal protein L41 which confers resistance against cycloheximide. Therefore, its applicability is limited, as it allows only for a single selection round after transformation with recombinant DNA.

It would be highly desirable to avail of a transformation system which can be repeatedly used and which allows for multiple events of genetic engineering, allowing Pichia ciferrii to be genetically modified in a rational manner, with the purpose to use this yeast for the production of low-molecular-weight compounds, such as sphingolipids, but also for the production of macromolecules, such as peptides or proteins.

In a first aspect, the present invention provides Pichia ciferrii cells defective in a biosynthetic pathway. The biosynthetic pathway may be a pathway for biosynthesis of an amino acid, a nucleotide and/or a vitamin. More preferably, the biosynthetic pathway is a pathway for biosynthesis of uracil, adenine, tryptophan, lysine and/or methionine. Most preferably, the biosynthetic pathway is a pathway for biosynthesis of uracil and/or lysine.

According to the invention, “defective in a biosynthetic pathway” means that a cell possessing that feature is not capable to synthesise the metabolite that is the product of that biosynthetic pathway. The Pichia ciferrii cells thereby may be defective in one or more biosynthetic pathways.

The Pichia ciferrii cells defective in a biosynthetic pathway were surprisingly obtained by simple selection of spontaneous mutants capable of growing in the presence of an otherwise toxic metabolite analogue.

The method to prepare the Pichia ciferrii cells defective in a biosynthetic pathway comprises culturing a population of Pichia ciferrii cells in the presence of a toxic metabolite analogue under conditions wherein the majority of cells is not capable to grow, and isolating clones that are able to grow.

If necessary, the amount, of mutants may be increased by subjecting the population of Pichia ciferrii cells to UV or chemical mutagenesis prior to the culturing in the presence of the toxic metabolite analogue.

A suitable concentration of the toxic metabolite analogue typically lies around the minimal inhibitory concentration (MIC value) found for the particular toxic metabolite analogue when cultured with the Pichia ciferrii cells. This concentration typically may be between, for instance, 1 and 10 mg/ml.

The toxic metabolite analogue to be used will depend on the biosynthetic pathway to be made defective. It may be 5-fluoroorotic acid, α-aminoadipic acid, 5-fluoroanthranilic acid, ureidosuccinic acid and/or methyl mercury, providing Pichia ciferrii cells that are defective in the metabolic pathway for uracil, lysine, tryptophan and/or methionine respectively. Preferably, the toxic metabolite analogue is 5-fluoroorotic acid and/or α-aminoadipic acid. Resistance of the provided Pichia ciferrii cells against these metabolite analogues is due to spontaneous (or induced if classical mutagenesis techniques are applied) loss-of-function mutations in the structural genes encoding those biosynthetic enzymes which would otherwise metabolize the metabolite analogues, resulting in toxicity to the cells.

Alternatively, mutant strains with loss-of-function mutations in a structural gene encoding a biosynthetic enzyme can be isolated by other means of enrichment of spontaneous auxotrophic mutants. For instance, enrichment of such spontaneous auxotrophic mutants can be performed by cultivation of a population of cells in a minimal medium containing substances which kill growing cells, such as nystatin. Consequently, cells with mutations resulting in an auxotrophic phenotype can not grow, survive and are thereby enriched. Auxotrophic mutants can subsequently be identified by testing their growth requirements.

Thus, the biosynthetic enzymes inactivated by loss-of-function mutations may be dihydroorotate dehydrogenase, orotidine-5′-phosphate decarboxylase, orotate phosphoribosyltransferase, tryptophan synthase, homoserine O-acetyltransferase, sulfite reductase, O-acetylhomoserine O-acetylserine sulfydrylase, and/or α-aminoadipate reductase. Preferably, the biosynthetic enzymes inactivated by loss-of-function mutations are orotidine-5′-phosphate decarboxylase or α-aminoadipate reductase. Inactivation of α-aminoadipate reductase may be due to loss-of-function mutations in the gene encoding α-aminoadipate reductase (LYS2) or may alternatively caused by loss-of-function mutations in the gene encoding α-aminoadipate reductase phosphopantetheinyl transferase (LYS5). α-Aminoadipate reductase phosphopantetheinyl transferase is required for post-translational modification of α-aminoadipate reductase, which is essential for α-aminoadipate reductase activity.

The Pichia ciferrii mutants defective in a biosynthetic pathway appear to display a stable auxotrophic phenotype, which means that they do not revert to a prototrophy upon prolonged culturing under non-selective conditions, or only show a low reversion frequency.

It is very surprising that spontaneous Pichia ciferrii mutants defective in a biosynthetic pathway and possessing a stable auxotrophic phenotype could be obtained. Successful selection of spontaneous or even UV-induced auxotrophic mutants has to our knowledge only been described for haploid yeast species such as Saccharomyces cerevisiae, Candida utilis (WO9814600) and Candida lusitanae (Young et al., A STE12 homolog is required for mating but dispensable for filamentation in Candida lusitaniae. Genetics. 2000. 155:17-29; Francois et al., Development of an integrative transformation system for the opportunistic pathogenic yeast Candida lusitaniae using URA3 as a selection marker. Yeast. 2004. 21:95-106), but not for diploid yeast species such as Pichia ciferrii (Wickerham and Burton, 1962, Bacteriol Rev. 26:382-397). Attempts to obtain spontaneous auxotrophic mutants of diploid yeast cells have repeatedly been described, but reportedly failed, even if the diploid yeast cells were mutagenized prior to selection for auxotrophic phenotypes (e.g. Wellington and Rustchenko, 5-Fluoroorotic acid induces chromosome alterations in Candida albicans. Yeast. 2005 Jan. 15;22(1):57-70.).

In fact, though the URA3 marker is used as selection marker in the diploid yeast Candida albicans, the required homozygous ura3/ura3 strains had to be created by laborious manipulations such as two rounds of targeted inactivation (Gorman et al., Repeated use of GAL1 for gene disruption in Candida albicans. Genetics. 1991. 129:19-24; Fonzi and Irwin, Isogenic strain construction and gene mapping in Candida albicans. Genetics. 1993. 134:717-28; Morschhäuser et al. Sequential gene disruption in Candida albicans by FLP-mediated site-specific recombination. Mol Microbiol. 32:547-56.) or by targeted inactivation via cotransformation of the deletion construct and a replicative plasmid containing the selection marker (Kelly et al., One-step gene disruption by cotransformation to isolate double auxotrophs in Candida albicans. Mol Gen Genet. 1988. 214:24-31.).

These laborious manipulations would not be easily applicable to Pichia ciferrii. They would either require the availability of two different dominant selection markers (sequential disruption of both alleles), conditional promoters functional in Pichia ciferrii (FLP recombinase-mediated marker rescue), or vectors capable of autonomous replication (co-transformation). However, none of these tools is available for Pichia ciferrii.

The Pichia ciferrii cells of the first aspect defective in a biosynthetic pathway may be host cells in a transformation system further comprising a polynucleotide comprising a functional gene that complements the defect in the biosynthetic pathway in which the Pichia ciferrii cells are defective.

In a second aspect there is provided a method of transforming a Pichia ciferrii cell of the first aspect having a defective biosynthetic pathway, comprising transforming the cell with a polynucleotide containing a functional gene that complements the defect in the biosynthetic pathway. Typically, the complementing functional gene encodes a functional enzyme of the biosynthetic pathway. The complementing functional gene may be any functional gene providing complementation of the defect in the biosynthetic pathway. It may be from any suitable origin, i.e. from bacterial, yeast, fungal, plant or animal species, but preferably is from a yeast. Most preferred is the use of a functional gene originating from Pichia ciferrii.

In a third aspect of the present invention there is provided a novel polynucleotide for use in a method of the previous aspect, wherein the polynucleotide contains a functional gene that complements the defect in said biosynthetic pathway in which the host cell is defective. The polynucleotide preferably is capable of integration into the genome of the Pichia ciferrii host cell.

In one embodiment of the invention, the complementing functional gene has a nucleotide sequence encoding a polypeptide with an amino acid sequence of SEQ ID NO:2, an amino acid sequence of SEQ ID NO:4, an amino acid sequence having a degree of identity of at least 91%, preferably at least 95%, to the amino acid sequence of SEQ ID NO:2 and/or an amino acid sequence having a degree of identity of at least 70%, preferably at least 80%, more preferably at least 90%, most preferably at least 95%, to the amino acid sequence of SEQ ID NO:4. Preferred functional genes have a nucleotide sequence according to SEQ ID NO:1, SEQ ID NO:3 and/or a sequence degenerate thereto.

The polynucleotide of this aspect may be in the form of a vector that may conveniently be subjected to recombinant DNA procedures and can be successfully introduced into Pichia ciferrii by transformation. The vector may be an autonomously replicating vector, i.e. a vector which exists as an extra-chromosomal entity, the replication of which is independent of chromosomal replication, e.g. a plasmid, cosmid, virus or phage vector, usually provided with an origin of replication. Preferably, the vector may be one which, when introduced into a host cell, is integrated into the host cell genome and replicated together with the chromosome(s) into which it has been integrated.

The vector may contain an expression cassette wherein the complementing functional gene is operably linked to regulatory sequences that are capable of providing for the expression of the encoded polypeptide by the Pichia ciferrii cells. Preferably, the expression cassette is flanked at its 5′ and 3′ sides by direct repeats of DNA. After the expression cassette flanked by direct repeats of DNA has been introduced into Pichia ciferrii cells by transformation, homologous recombination between the direct repeats of DNA may occur, leading to deletion of the selection marker contained in the expression cassette. This enables the use of Pichia ciferrii for sequential rounds of transformation. Cells that have lost the selection marker may be selected for by using the toxic metabolite analogue as described above. The direct repeats of DNA flanking the expression cassette typically have a length of at least 20 bp, preferably at least 100 bp, and have a degree of identity of at least 80% over their whole length. They may be derived from Pichia ciferrii or from another yeast or any other suitable source. Alternatively, they may be synthetically produced polynucleotides.

In a fourth aspect, the present invention provides novel polypeptides that are able to complement a defect in a biosynthetic pathway of Pichia ciferrii.

In one embodiment a polypeptide is provided that complements a defect in the uracil biosynthetic pathway and has an amino acid sequence according to SEQ ID NO: 2 or an amino acid sequence displaying a degree of identity of at least 91%, preferably at least 95%, to the amino acid sequence of SEQ ID NO:2. In another embodiment a polypeptide is provided that complements a defect in the lysine biosynthetic pathway and has an amino acid sequence according to SEQ ID NO: 4 or an amino acid sequence having a degree of identity of at least 70%, preferably at least 80%, more preferably at least 90%, most preferably at least 95%, to the amino acid sequence of SEQ ID NO: 4.

In a further aspect the invention provides a method for preparing a compound of interest using Pichia ciferrii cells defective in a biosynthetic pathway. The method comprises transforming Pichia ciferrii cells of the first aspect of this invention, i.e. defective in a biosynthetic pathway, with a polynucleotide of the third aspect (the selection marker) and a polynucleotide comprising a DNA sequence of interest, selecting transformed cells for prototrophy, culturing transformed cells under conditions conducive to production of the compound of interest, and, optionally, recovering the compound of interest.

The polynucleotides comprising the selection marker and the DNA sequence of interest may be contained in one DNA molecule or may be separate DNA molecules.

If the compound of interest is a polypeptide, the DNA sequence of interest typically encodes the polypeptide of interest. If the polypeptide is produced as a secreted protein, the nucleotide sequence encoding a mature form of the polypeptide in the expression construct is operably linked to a nucleotide sequence encoding a signal peptide.

If the compound of interest is a metabolite, e.g. a sphingoid base or other sphingolipids, the DNA sequence of interest may be a sequence suitable for metabolic engineering. For instance, the DNA sequence of interest may encode an enzyme of the metabolic pathway leading to production of the compound of interest. It is also possible that the DNA sequence of interest is intended to inactivate a gene encoding an enzyme activity that is undesirable for production of the compound of interest.

### BRIEF DESCRIPTION OF THE FIGURES

FIG. 1 schematically describes the four-step procedure resulting in the isolation of the entire Pichia ciferrii URA3 locus.

Amplification of an internal part of PcURA3 (I.) was followed by three rounds of inverse PCR (II. to IV.) Oligonucleotides used in the individual steps are indicated and sequence representations in different shadings show the portions of the PcURA3 locus whose DNA sequence were determined in the individual steps. Restriction sites relevant for the experimental procedures are also indicated.

FIG. 2 schematically describes the three-step procedure resulting in the isolation of the entire Pichia ciferrii LYS2 locus.

Amplification of an internal part of PcLYS2 (I.) was followed by two rounds of inverse PCR (II. and III.) Oligonucleotides used in the individual steps are indicated and sequence representations in different shadings show the portions of the PcLYS2 locus whose DNA sequence were determined in the individual steps. Restriction sites relevant for the experimental procedures are also indicated.

FIG. 3 shows a graphical representation of the plasmid pDB008 for complementation of uracil-auxotrophic Pichia ciferrii mutants with the Pichia ciferrii URA3 gene. The PcURA3 promoter (white box), the PcURA3 coding sequence (black arrow), the PcURA3 terminator (horizontally hatched box), the ampicillin resistance gene (bla; light grey arrow) and the 5S-26S rDNA intergenic spacer with internal Pmel recognition site (IS; gridded box) are shown. Restriction sites relevant for the cloning procedures are also indicated.

FIG. 4 shows a graphical representation of the plasmid pDB012 for complementation of lysine-auxotrophic Pichia ciferrii mutants with the Pichia ciferrii LYS2 gene. The Pc LYS2 promoter (white box), the Pc LYS2 coding sequence (black arrow), the Pc LYS2 terminator (horizontally hatched box), the ampicillin resistance gene (bla; light grey arrow) and the 5S-26S rDNA intergenic spacer with internal Pmel recognition site (IS; gridded box) are shown. Restriction sites relevant for the cloning procedures are also indicated.

### EXAMPLES

### Example 1

Isolation of Genomic DNA from Pichia ciferrii F-60-10A NRRL 1031

Pichia ciferrii F-60-10A NRRL 1031 was grown in 50 ml YEPD medium (peptone 2% (w/v), yeast extract 1% (w/v) and glucose 2% (w/v) in 250 ml Erlenmeyer flasks at 200 rpm and 30° C. and harvested after 18 h to an OD₆₀₀ of 1.5. Chromosomal DNA was isolated using the PUREGENE® DNA Purification Kit for Yeast and Gram-positive bacteria (Gentra Systems Inc., cat.# D-6000A) according to the instructions of the manufacturer. A quality check of the isolated DNA by agarose gel electrophoresis demonstrated its high molecular weight (>16 kbp).

### Example 2

Isolation of Uracil-Auxotrophic Mutants of Pichia ciferrii F-60-10A NRRL 1031

Mutations at the URA3 locus of Saccharomyces cerevisiae can be obtained by a positive selection. Wild-type strains of yeast are unable to grow on medium containing the pyrimidine analog 5-fluoroorotic acid (5-FOA), whereas ura3 mutants grow normally (Boeke et al. A positive selection for mutants lacking orotidine-5′-phosphate decarboxylase activity in yeast: 5-fluoro-orotic acid resistance. Molecular & General Genetics, 197: 345-346, 1984 and Boeke et al. 5-Fluoroorotic acid as a selective agent in yeast molecular genetics. Methods in Enzymology, 154: 164-175, 1987). Orotidine-5′-phosphate decarboxylase, encoded by URA3, converses 5-FOA to fluorodeoxyuridin, which serves as a toxic agent in yeast. In presence of uracil ura3 mutants are able to grow on medium containing 5-FOA.

To select ura3 mutants Pichia ciferrii F-60-10A NRRL 1031 was grown in 50 ml YEPD medium (peptone 2.0% (w/v), yeast extract 1.0% (w/v) and glucose 2.0% (w/v)) in 250 ml Erlenmeyer flasks at 250 rpm and 30° C. to an OD₆₀₀ of 10.0, which corresponds to a titer of 1×10⁸ cells/ml. 5×10⁷ cells were plated on solid 5-FOA medium (glucose 4.0% (w/v), agar-agar 2.0% (w/v), yeast nitrogen base without amino acids 0.67% (w/v), uracil 2.0% (w/v) and 5-FOA 1.0% (w/v)) and incubated for 6 days at 30° C. About 50 colonies per plate appeared after the described incubation. They were analyzed with respect to uracil auxotrophy by transferring them to solid YNB medium without uracil (glucose 2.0% (w/v), agar-agar 2.0% (w/v), yeast nitrogen base without amino acids 0.67% (w/v)). Clones showing no growth on solid YNB medium without uracil were inoculated in 5 ml YEPD medium in test tubes to test for reversion of uracil auxotrophy in non-selective medium. After 3 days of growth at 250 rpm and 30° C. the cultures reached stationary phase. 100 μl (3×10⁷ cells) of these cultures were plated on YNB medium lacking uracil. Those clones showing no growth (no reversion to uracil prototrophy) on solid YNB medium lacking uracil were referred to as uracil auxotrophic Pichia ciferrii strains lacking orotidine-5′-phosphate decarboxylase activity.

### Example 3

Cloning and Determination of the Nucleotide Sequence of the Pichia ciferrii URA3 Gene

Amplification of an Internal Part of the Pichia ciferrii URA3 Gene

First, the amino acid sequences of putative orotidine-5′-phosphate decarboxylases from Saccharomycotina species were extracted from NCBI's database of completed and unfinished eukaryotic genomes (www.ncbi.nlm.nih.gov/sutils/genom_table.cgi) by performing a BLASTP search with the Saccharomyces cerevisiae Ura3p (GenBank acc.# NP_{—}010893) as template. This protein has been characterized biochemically and been proven to represent an orotidine-5′-phosphate decarboxylase (Bell and Jones. Purification and characterization of yeast orotidine 5′-monophosphate decarboxylase overexpressed from plasmid PGU2. The Journal of Biological Chemistry, 266:12662-12667, 1991). The extracted sequences (all entries with E-values<6×10⁻⁷¹) were aligned using the ClustalW program (www.ebi.ac.uk/clustalw). Suitable oligonucleotides for amplification of an internal part of the Pichia ciferrii URA3 gene were derived by back-translating highly conserved stretches of amino acids within the Ura3p sequence taking into account the highly biased Pichia ciferrii codon usage. The following oligonucleotides were then synthesized by MWG Biotech (Ebersberg, Germany):

| | |
|---|---|
| PcURA3-fw1: | |
| TTY GAA GAT MGW AAA TTY GCW | (nt 1339-1359 in |
| | SEQ ID NO: 1) |
| and | |
| PcURA3-rv1: | |
| AAY AAA CCW CKA CCR ACR ATR AT. | (nt 1787-1765 in |
| | SEQ ID NO: 1) |

These oligonucleotides were used to set up a PCR reaction according to Innis et al., (PCR protocols. A guide to methods and applications, 1990, Academic Press) with Herculase® Hot Start PCR Master Mix (Stratagene, cat.# 600612) according to the instructions of the manufacturer and chromosomal DNA from Pichia ciferrii F-60-10A NRRL 1031 as template. A 449 bp fragment could be obtained by applying this method. The fragment was purified using the MinElute Gel Extraktion Kit (Qiagen, cat.# 28606) according to the instructions of the manufacturer.

Determination of the DNA Sequence of an Internal Part of the Pichia ciferrii URA3 Gene

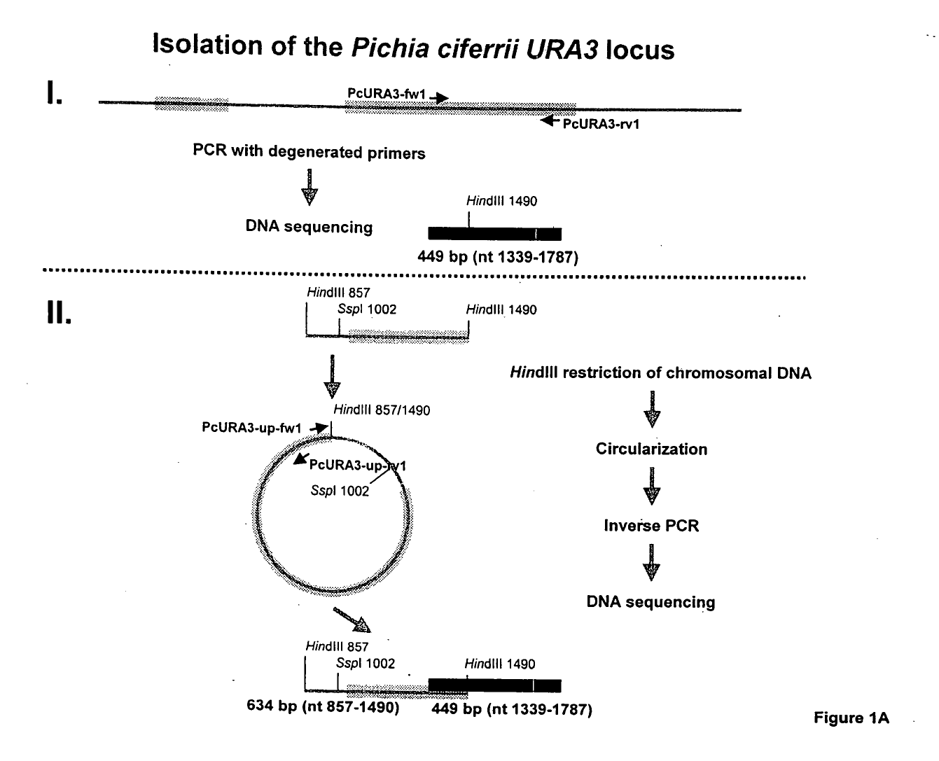

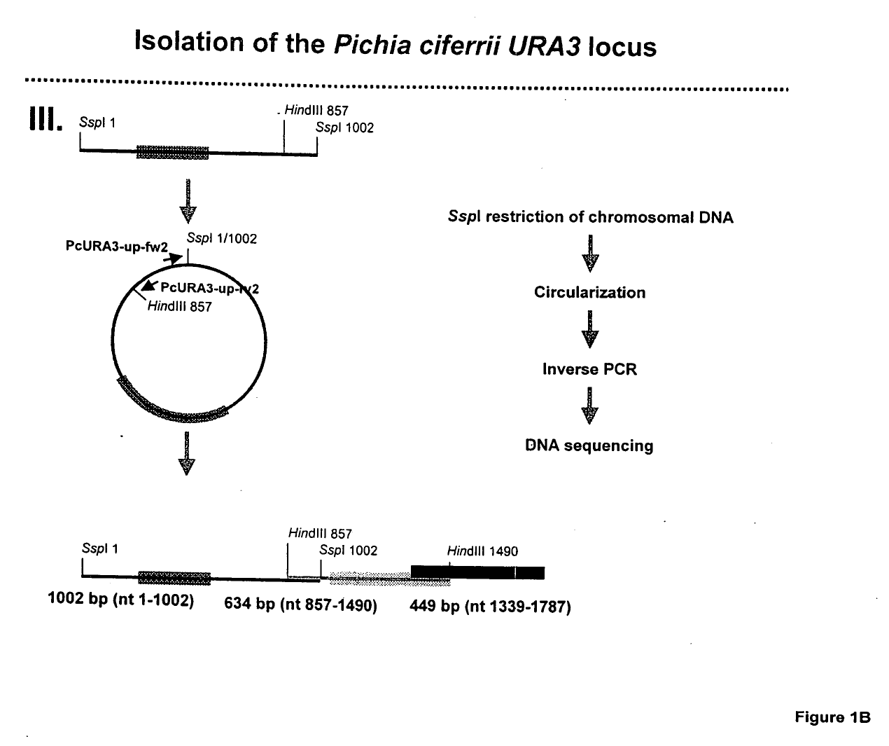

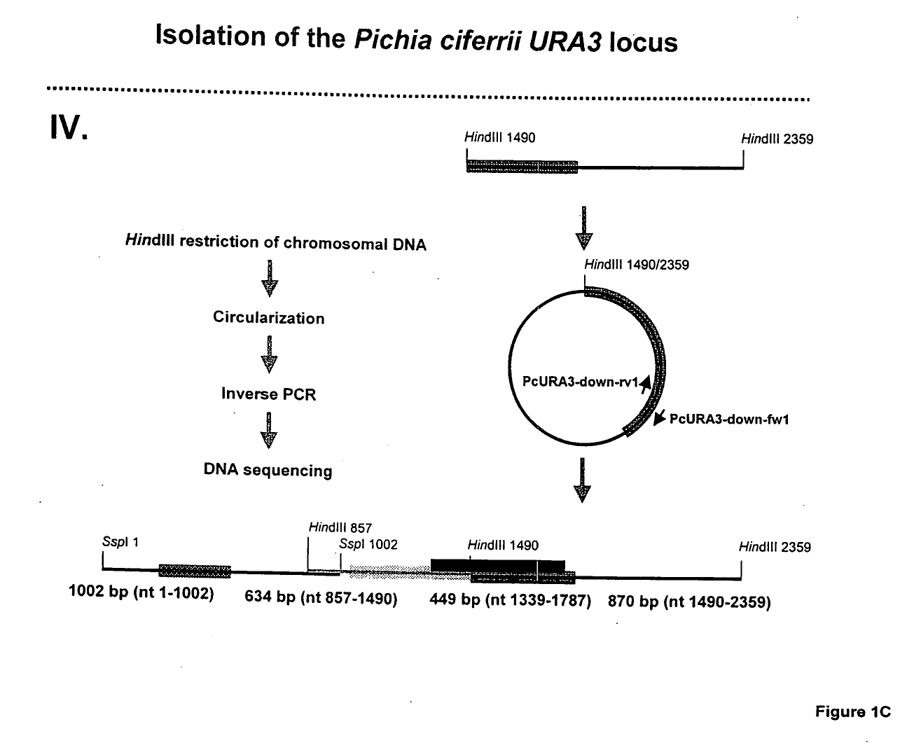

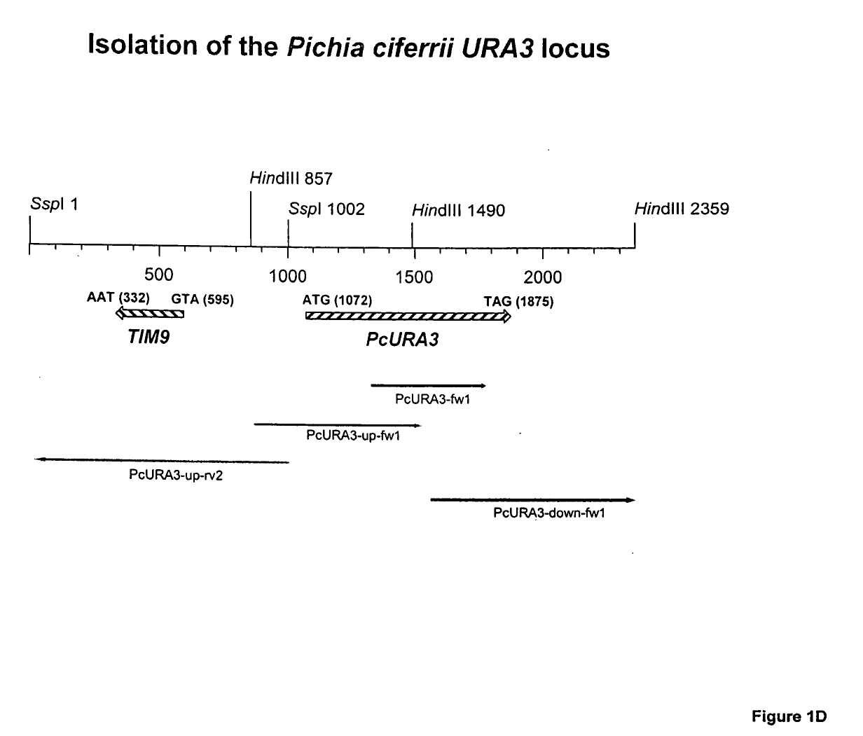

The DNA sequence of the purified PCR product was determined using the dideoxy chain termination method developed by Sanger et al. (Proceedings of the National Academy of Sciences, U.S.A., 74:5463-5467). As sequencing primer PcURA3-fw1 was used. DNA sequencing was performed by Sequiserve (Vaterstetten, Germany). The generated sequence information (449 bp, corresponding to nt 1339-1787 in SEQ ID NO: 1; FIG. 1) was translated into protein using the Clone Manager 7 software (Scientific & Educational Software) and the resulting amino acid sequence used as template for a BLASTP search with NCBI's non-redundant protein database (www.ncbi.nlm.nih.gov/BLAST/). The search resulted in the identification of Kluyveromyces lactis Ura3p (NCBI acc.# XP_{—}454981) as being the protein in the database most similar to the new sequence, confirming that in fact portions of the Pichia ciferrii URA3 ortholog had been amplified.

Amplification of the Entire Pichia ciferrii URA3 Gene and Determination of its DNA Sequence

In order to determine the DNA sequence of the entire Pichia ciferrii URA3 gene (coding sequence, promoter region and 3′-untranslated region) an inverse PCR approach was followed. Chromosomal DNA (300 ng) from Pichia ciferrii F-60-10A NRRL 1031 (isolated as described in Example 1) was digested overnight with HindIII (New England Biolabs, cat.# R0104L) according to the instructions of the manufacturer in a total volume of 50 μl. The digested DNA was purified using the QIAquick PCR Purification Kit (Qiagen, cat.# 28106) according to the instructions of the manufacturer. The eluted DNA (50 μl) was subjected to overnight ligation using the T4 DNA Ligase (New England Biolabs, cat.# M0202L) according to the instructions of the manufacturer in a total volume of 200 μl with 800 U of T4 DNA Ligase. The ligated DNA was purified using the QIAquick PCR Purification Kit according to the instructions of the manufacturer. 2.5 μl of the eluate was used as template for an inverse PCR reaction according to Innis et al., (PCR protocols. A guide to methods and applications, 1990, Academic Press). To that end two oligonucleotides hybridizing with the already known portion of the Pichia ciferrii URA3 gene were used:

| | |
|---|---|
| PcURA3-up-fw1: | |
| AAT ATG CAG GTG GTG CTT TCA AAA TT | (nt 1385-1410 in |
| | SEQ ID NO: 1) |
| and | |
| PcURA3-up-rv1: | |
| GTG CTT TGA CTG TGT TCC CTA T. | (nt 1384-1363 in |
| | SEQ ID NO: 1) |

Amplification was performed with Phusion™ High Fidelity PCR Master Mix according to the instructions of the manufacturer. Using this procedure a 0.6 kbp PCR product could be obtained. The fragment was purified using the MinElute PCR Purification Kit (Qiagen, cat.# 28006) according to the instructions of the manufacturer. The DNA sequence of this fragment was determined as described previously, using oligonucleotide PcURA3-up-fw1 as sequencing primer. The newly obtained sequence information covered nt 857-1338 in SEQ ID NO: 1. In order to obtain further information about the upstream region of the Pichia ciferrii URA3 another round of inverse PCR had to be performed. Therefore, the above described experimental protocol was repeated, except that SspI (New England Biolabs, cat.# R0132S) was used for digesting Pichia ciferrii chromosomal DNA and the following oligonucleotides, synthesized by MWG Biotech (Ebersberg, Germany), were employed during inverse PCR:

| | |
|---|---|
| PcURA3-up-fw2: | |
| CTT GTA CGG GAC ACG CTT CTA TG | (nt 953-975 in |
| | SEQ ID NO: 1) |
| and | |
| PcURA3-up-rv2: | |
| TCT ATG GGA CGC CTA GTG TCT C. | (nt 888-867 in |
| | SEQ ID NO: 1) |

Using this procedure a 1.0 kbp PCR product could be obtained. The fragment was purified using the MinElute PCR Purification Kit according to the instructions of the manufacturer. The DNA sequence of this fragment was determined as described previously with the oligonucleotide PcURA3-up-rv2 as sequencing primer. 856 bp of new sequence information (nt 1-856 in SEQ ID NO: 1) could be obtained which stretches to the next SspI site (FIG. 1). No new sequence information downstream of the already known DNA sequence could be obtained as the primers used for inverse PCR anneal upstream of the SspI site (FIG. 1). In order to obtain the DNA sequence of the 3′-end of the coding region of the Pichia ciferrii URA3 gene and its 3′-untranslated region another round of inverse PCR had to be performed. Therefore, the above described experimental protocol was repeated, except that HindIII (New England Biolabs, cat.# R0104L) was used for digesting Pichia ciferrii chromosomal DNA and the following oligonucleotides, synthesized by MWG Biotech (Ebersberg, Germany), were employed during inverse PCR:

| | |
|---|---|
| PcURA3-down-fw1: | |
| GTT GTT TCA ACT GGT AGT GAT ATC AT | (nt 1741-1766 in |
| | SEQ ID NO: 1) |
| and | |
| PcURA3-down-rv1: | |
| GGT ATC AAC GGA TCT ATA TTG TTG A. | (nt 1740-1716 in |
| | SEQ ID NO: 1) |

Using this procedure a 0.9 kbp PCR product could be obtained. The fragment was purified using the MinElute PCR Purification Kit according to the instructions of the manufacturer. The DNA sequence of this fragment was determined as described previously with the oligonucleotide PcURA3-down-fw1 as sequencing primer. 572 bp of new sequence information (nt 1788-2359 in SEQ ID NO: 1) could be obtained which stretches to the next HindIII site (FIG. 1). Using the described three-step procedure, a total of 2359 bp of the Pichia ciferrii URA3 locus could be isolated and its DNA sequence be determined (see SEQ ID NO: 1 and FIG. 1).

The Pichia ciferrii URA3 locus as depicted in FIG. 1 encodes the PcUra3p protein of 267 amino acids in length (SEQ ID NO: 2). PcUra3p has 76% (91%) and 78% (87%) positional amino acid identity (similarity) to hypothetical orotidine-5′-phosphate decarboxylases from Debaromyces hansenii (GenBank acc.# XP_{—}456850) and Candida glabrata (GenBank acc.# XP_{—}447384), respectively. Additionally the PcUra3p protein shows 75% identity and 89% similarity to Candida albicans orotidine-5′-phosphate decarboxylase, which has been shown to complement Saccharomyces cerevisiae ura3 mutations (Losberger and Ernst. Sequence and transcript analysis of the C. albicans URA3 gene encoding orotidine-5′-phosphate decarboxylase. Current Genetics, 16: 153-158, 1989).

### Example 4

Construction of a Vector for Complementation of Uracil-Auxotrophic Pichia ciferrii Strains

In order to complement the uracil auxotrophy of the uracil auxotrophic Pichia ciferrii strain (see Example 2) we constructed an integrative URA3 expression vector.

To that end 200 ng of chromosomal DNA of Pichia ciferrii F-60-10A NRRL 1031 (isolated as described in Example 1) was used as template for a PCR according to Innis et al. (PCR protocols. A guide to methods and applications, 1990, Academic Press) to amplify the promotor region of glyceraldehyde-3-phospate dehydrogenase of Pichia ciferrii (TDH1). For this the following oligonucleotides were applied:

pGAP-BglII-for:
5′-TAT ATA AGA TCT GTG GTA CCT ACA TAC AAT TGA
CCC-3′ (including a BglII-recognition sequence at
the 5′ end)
and
pGAP-NcoI-rev:
5′-TAT ATA CCA TGG TTA ATT AAT TAT TTG TTT GTT TG-
3′ (including a NcoI-recognition sequence at the
5′ end).

The fragment was purified using the QIAquick PCR Purification Kit according to the instructions of the manufacturer. Then digestion of the PCR product with BglII and NcoI (according to the instructions of the manufacturer of the restriction endonuclease: New England Biolabs, Schwalbach, Germany) yielded a 575 bp fragment, which was ligated into respectively cut pAG25 (Goldstein et al., Three new dominant gene disruption cassettes for gene disruption in Saccharomyces cerevisiae. Yeast, 15:1541-1553, 1999) creating vector pTH-GAP-nat1 (3892 bp) with the promotor region of glyceraldehyde-3-phospate dehydrogenase gene (TDH3) of Pichia ciferrii fused to nat1. The orientation and authenticity of the insert was determined by DNA sequencing. Ligation, preparation and transformation of chemically competent Escherichia coli cells was performed by methods known to the skilled person.

As the 5S-26S rDNA intergenic spacer (IS) of Pichia ciferrii was to be used as integration site for the plasmid, portions of the 5S-26S rDNA intergenic spacer (IS) were amplified by PCR using 200 ng of chromosomal DNA of Pichia ciferrii F-60-10A NRRL 1031 as template and following oligonucleotides:

pIS-NdeI-for:
5′-TAT ATA CAT ATG CTA ATC ACA ACA GAA CAT TCT CTA
ACG-3′ (including a NdeI-recognition sequence at
the 5′ end)
and
pIS-NdeI-rev:
5′-TAT ATA CAT ATG GCT AGA TTG ACA GAA GTC GAT
CAG-3′ (including a NdeI-recognition sequence at
the 5′ end).

The fragment was purified using the QIAquick PCR Purification Kit according to the instructions of the manufacturer. Vector pTH-GAP-nat1 and the PCR product were digested with NdeI (according to the instructions of the manufacturer of the restriction endonuclease: New England Biolabs, Schwalbach, Germany) followed by ligation, creating vector pTH-GAP-nat1-IS2 (4864 bp). The orientation and authenticity of the insert was determined by DNA sequencing. Ligation, preparation and transformation of chemically competent Escherichia coli cells was performed by methods known to the skilled person.

To insert a single PmeI-recognition sequence for linearization of vector pTH-GAP-nat1-IS2, two fragments of the 5S-26S rDNA intergenic spacer (IS) of the Pichia ciferrii ribosomal RNA operon integrated into pTH-GAP-nat1-IS2 were amplified by PCR using vector pTH-GAP-nat1-IS2 as template. Fragment 1 was amplified using oligonucleotides:

pIS-NdeI-rev:
5′-TAT ATA CAT ATG GCT AGA TTG ACA GAA GTC GAT
CAG-3′ (including a NdeI-recognition sequence at
the 5′ end)
and
PmeI-rv:
5′-CCC ATC CAC TAA GTT TAA ACA CCC ATA CAA AAT CGA
GCT TCA AAT C-3′ (including a 21 bp sequence com-
plementary to the oligonucleotide PmeI-fw and a
PmeI-recognition sequence at the 5′ end).

Fragment 2 was amplified using oligonucleotides:

p-IS-NdeI-for:
5′-TAT ATA CAT ATG CTA ATC ACA ACA GAA CAT TCT CTA
ACG-3′ (including a NdeI-recognition sequence at
the 5′ end)
and
PmeI-fw:
5′-TGT TTA AAC TTA GTG GAT GGG AAA CCC TGT AGA ACT
GGA CAA AC 3′ (including a PmeI-recognition
sequence at the 5′ end).

The fragments were purified using the QIAquick PCR Purification Kit according to the instructions of the manufacturer. Finally, a fusion of fragments 1 and 2 was obtained by setting up a PCR with 10 ng of each of the two primary PCR products as templates with oligonucleotides:

p-IS-NdeI-for:
5′-TAT ATA CAT ATG CTA ATC ACA ACA GAA CAT TCT CTA
ACG-3′ (including a NdeI-recognition sequence at
the 5′ end)
and
pIS-NdeI-rev:
5′-TAT ATA CAT ATG GCT AGA TTG ACA GAA GTC GAT
CAG-3′ (including a NdeI-recognition sequence at
the 5′ end)

generating a 978 bp fragment with Ndel-recognition sequences at both ends and a PmeI-recognition sequence in the middle of the fragment.

The fragment was purified using the QIAquick PCR Purification Kit according to the instructions of the manufacturer. PCR product and vector pTH-GAP-nat1-IS2 were cut with NdeI (according to the instructions of the manufacturer of the restriction endonuclease: New England Biolabs, Schwalbach, Germany). Ligation was performed to generate vector pTH-GAP-nat1-IS2-PmeI (4879 bp). The orientation and authenticity of the insert was determined by DNA sequencing. Ligation, preparation and transformation of chemically competent Escherichia coli cells was performed by methods known to the skilled person.

To introduce the Pichia ciferrii URA3 gene including the native promoter and terminator the URA3 gene was amplified using 200 ng of chromosomal DNA of Pichia ciferrii F-60-10A NRRL 1031 as template for a PCR with the following oligonucleotides:

PcURA3-SalI-fw:
5′-TAT GTC GAC TTT GAT TGA TTT GAT ATA GAT TTG-3′
(including a SalI-recognition sequence at the 5′
end)
and
PcURA3-rv:
5′-TGC TGG AGG GGT TCT TAA GTA TAT AG-3′.

The resulting 1752 bp fragment was purified using the MinElute PCR Purification Kit according to the instructions of the manufacturer. Then the PCR product was subjected to digestion with the restriction endonucleases SalI and SacI (according to the instructions of the manufacturer of the restriction endonucleases: New England Biolabs, Schwalbach, Germany). The digested 1703 bp fragment was gel-purified using the MinElute Gel Extraktion Kit according to the instructions of the manufacturer and ligated into the vector backbone of pTH-GAP-nat1-IS2-PmeI, which had been digested with SalI and SacI and been gel-purified (3448 bp), to generate vector pDB008 (5172 bp). The orientation and authenticity of the insert was determined by DNA sequencing. Ligation, preparation and transformation of chemically competent Escherichia coli cells was performed by methods known to the skilled person.

### Example 5

Complementation of Uracil-auxotrophic Pichia ciferrii Strains with Plasmid pDB008

To complement uracil-auxotrophic Pichia ciferrii cells, they were grown in YEPD medium to an OD₆₀₀ of 1 to 1.5. The cells were harvested by centrifugation and resuspended in 0.1 culture volume of 50 mM phosphate buffer (pH 7.5) to which 25 mM dithiothreitol was added. After incubation at 37° C. for 15 min, the cells were washed twice with one culture volume of cold stabilization solution (270 mM sucrose, 10 mM Tris-HCl [pH 7.5], 1 mM MgCl₂), and resuspended in 0.01 culture volume of stabilization solution. 2.5 μl vector pDB008 linearized by digestion with PmeI (see Example 4) (containing 1.6 μg DNA) or, as a negative control, 2.5 μl H₂0 were mixed with 50 μl of cell suspension and incubated on ice for 10 min. Then the transformation mixture was transferred to a 2 mm electroporation cuvette. Electroporation was performed with a GenePulser Xcell at 500 V, 50 μF and 700 Ω (Bio-Rad Laboratories, Hercules, U.S.A.). After electroporation the cells were resuspended in 500 μl of stabilization solution and transferred to a culture tube containing 2 ml YPD medium. After overnight regeneration at 30° C. and 250 rpm, aliquots of the regenerated cells were plated on YNB medium lacking uracil and incubated at 30° C. While no colonies appeared on plates containing the cells treated with water only, transformation of uracil-auxotrophic Pichia ciferrii cells with linearized vector pDB008 did result in formation of colonies on plates containing these cells after three days of incubation. Single colonies were analyzed for correct integration of plasmid pBD008 into the 5S-26S rDNA intergenic spacer via colony PCR. To that end the following oligonucleotides were synthesized by MWG Biotech (Ebersberg, Germany):

PcL41-up:
GCG TTC TAA CGA CAA TAT GTC C
and
26S rDNA-down:
GTT TGT GTT TAT CAC AAC ACT TCT AAG.

These oligonucleotides were used to set up a PCR reaction according to Innis et al., (PCR protocols. A guide to methods and applications, 1990, Academic Press) with Taq PCR Master Mix (Qiagen, cat.# 201445) according to the instructions of the manufacturer and cells from uracil-prototrophic colonies as template. Only in case of correct integration of pDB008 in the 5S-26S rDNA intergenic spacer, the PCR reaction would yield a 1040 bp fragment, demonstrating that integration of vector pDB008 harbouring Pichia ciferrii URA3 gene did take place at the 5S-26S rDNA intergenic spacer of Pichia ciferrii. All colonies tested led to amplification of the 1040 bp fragment. Consequently, transformation of uracil-auxotrophic Pichia ciferrii cells with vector pDB008 containing the Pichia ciferrii URA3 gene resulted in complementation of the biosynthetic defect.

### Example 6

Isolation of Lysine Auxotrophic Mutants of Pichia ciferrii F-60-10A NRRL 1031

In contrast to wild-type strains of the yeast Saccharomyces cerevisiae, certain lys mutants are able to utilize α-aminoadipate as a primary source of nitrogen (Chattoo et al. α-Aminoadipate as a primary nitrogen source for Saccharomyces cerevisiae mutants. Genetics, 93: 51-65, 1979). The anabolism of high levels of α-aminoadipate through the biosynthetic pathway of lysine results in the accumulation of α-aminoadipic-δ-semialdehyde which serves as a toxic intermediate. The accumulation of this toxic intermediate is prevented by lys2 and lys5 mutations (Zaret and Sherman, Journal of Bacteriology, 162: 579-583, 1985) which both result in an inactive α-aminoadipate reductase enzyme.

To select lys mutants Pichia ciferrii F-60-10A NRRL 1031 was grown in 50 ml YEPD medium (peptone 2.0% (w/v), yeast extract 1.0% (w/v) and glucose 2.0% (w/v)) in 250 ml Erlenmeyer flasks at 250 rpm and 30° C. to an OD₆₀₀ of 1.1, which corresponds to a titer of 1.1×10⁷ cells/ml. 2.2×10⁷ cells were plated on solid α-aminoadipate medium (glucose 2.0% (w/v), agar-agar 2.0% (w/v), yeast nitrogen base without amino acids and without nitrogen source 0.167% (w/v), L-lysine 3.0% (w/v) and D,L-α-aminoadipate 0.15% (w/v)) and incubated for 6 days at 30° C. Several hundred α-aminoadipate-resistant colonies appeared after the described incubation. They were analyzed with respect to lysine auxotrophy by transferring them to solid YNB medium without lysine (glucose 2.0% (w/v), agar-agar 2.0% (w/v), yeast nitrogen base without amino acids 0.67% (w/v)). Clones showing no growth on solid YNB medium without lysine were inoculated in 5 ml YEPD medium in test tubes to test for phenotypic reversion of lysine auxotrophy in non-selective medium. After 4 days of growth at 250 rpm and 30° C. cultures reached stationary phase. 100 μl of stationary cultures were used to inoculate new test tubes with 5 ml YEPD medium. After incubation of these cultures for 2 days at 250 rpm and 30° C., 200 μl were plated on solid YNB medium lacking lysine. Those clones showing no growth (no reversion to lysine prototrophy) on solid YNB medium lacking lysine were referred to as lysine auxotrophic Pichia ciferrii strains lacking α-aminoadipate reductase activity. The lack in these Pichia ciferrii strains of α-aminoadipate reductase activity may be caused by a loss-of-function mutation in the gene encoding α-aminoadipate reductase, LYS2, or by a loss-of-function mutation in the gene encoding α-aminoadipate reductase phosphopantetheinyl transferase, LYS5. The latter enzyme is required for post-translational modification of α-aminoadipate reductase, which is essential for α-aminoadipate reductase activity.

### Example 7

Cloning and Determination of the Nucleotide Sequence of the Pichia ciferrii LYS2 Gene

Amplification of an Internal Part of the Pichia ciferrii LYS2 Gene

First, the amino acid sequences of putative α-aminoadipate reductases from Saccharomycotina species were extracted from NCBI's database of completed and unfinished eukaryotic genomes (www.ncbi.nlm.nih.gov/sutils/genom_table.cgi) by performing a BLASTP search with the Saccharomyces cerevisiae Lys2p (GenBank acc.# NP_{—}009673) as template. The corresponding gene has been identified to represent the structural gene for α-aminoadipate reductase (Sinha and Bhattacharjee. Lysine biosynthesis in Saccharomyces. Conversion of α-aminoadipate into α-aminoadipic-δ-semialdehyde. The Biochemical Journal, 125: 743-749, 1971), which catalyzes an essential step in the pathway for lysine biosynthesis in Saccharomyces cerevisiae and other fungi. The extracted sequences (all entries with E-values <6×10⁻⁷³) were aligned using the ClustalW program (www.ebi.ac.uk/clustalw). Suitable oligonucleotides for amplification of an internal part of the Pichia ciferrii LYS2 gene were derived by back-translating highly conserved stretches of amino acids within the Lys2p sequence taking into account the highly biased Pichia ciferrii codon usage. The following oligonucleotides were then synthesized by MWG Biotech (Ebersberg, Germany):

| | |
|---|---|
| PcLYS2-fw: | |
| GTY ATY GAT CCW GCW TAY CCW CC | (nt 1794-1816 in |
| | SEQ ID NO: 3) |
| and | |
| PcLYS2-rv: | |
| CCC AYT TWS WTT GAC CRT AAC C. | (nt 4293-4272 in |
| | SEQ ID NO: 3) |

These oligonucleotides were used to set up a PCR reaction according to Innis et al., (PCR protocols. A guide to methods and applications, 1990, Academic Press) with Phusion™ High Fidelity PCR Master Mix (Finnzymes, cat.# F-531L) according to the instructions of the manufacturer and chromosomal DNA from Pichia ciferrii F-60-10A NRRL 1031 as template. A 2500 bp fragment could be obtained by applying this method. The fragment was purified using the MinElute Gel Extraktion Kit (Qiagen, cat.# 28606) according to the instructions of the manufacturer.

Determination of the DNA Sequence of an Internal Part of the Pichia ciferrii LYS2 Gene

The DNA sequence of the purified PCR product was determined using the dideoxy chain termination method developed by Sanger et al. (Proceedings of the National Academy of Sciences, U.S.A., 74:5463-5467). As sequencing primers PcLYS2-fw, PcLYS2-rv and

| | |
|---|---|
| PcLYS2-intrv: | |
| CTG AAT TGA GAA TCA TCT TGG | (nt 3361-3341 in |
| | SEQ ID NO: 3) |

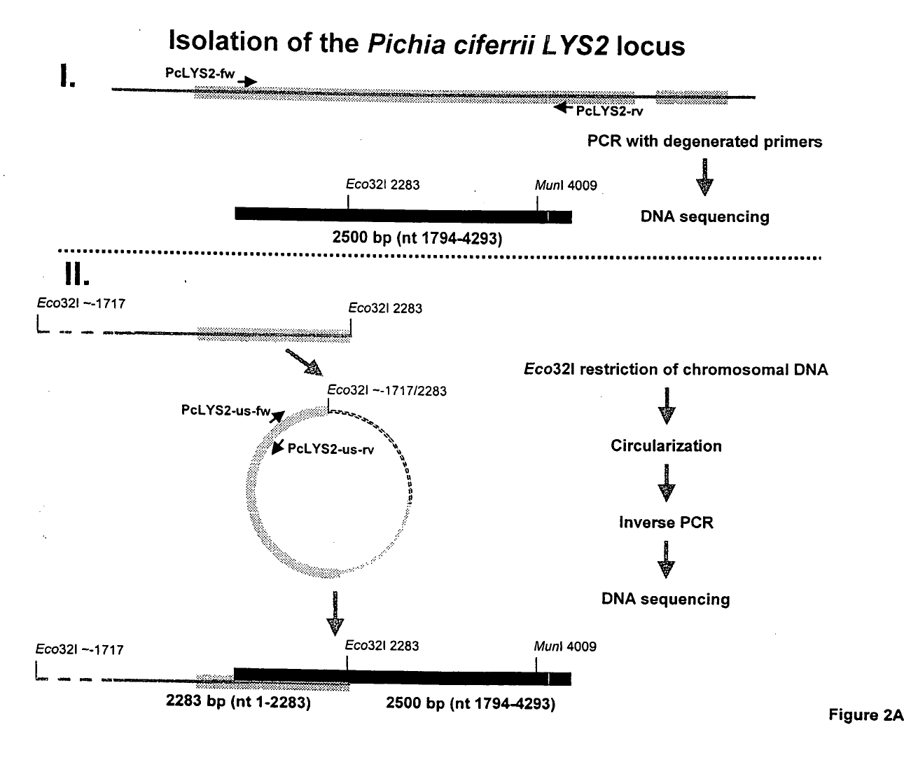

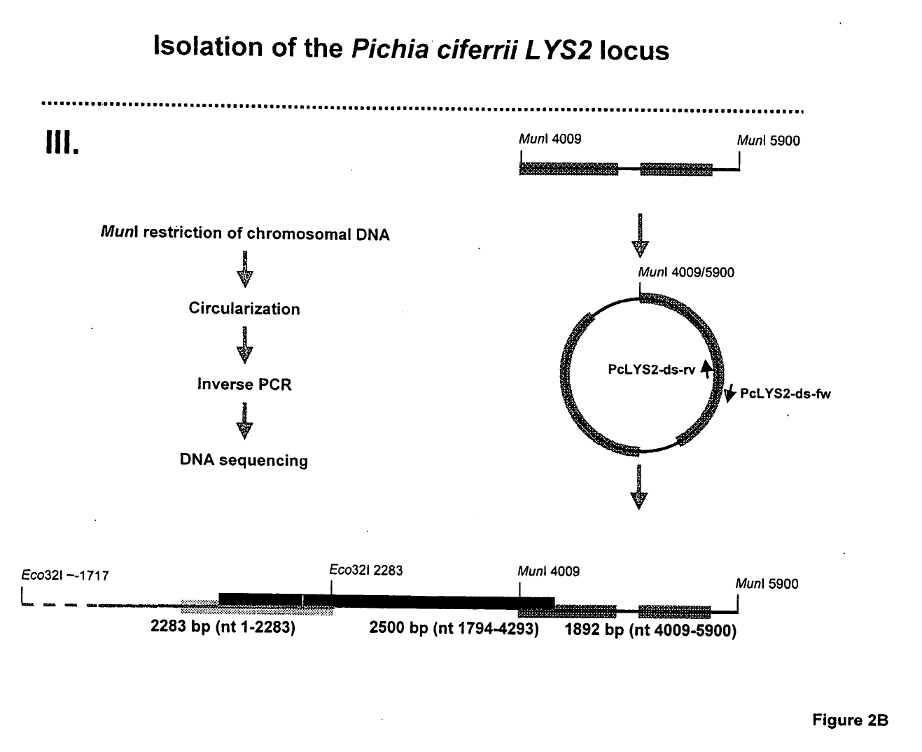

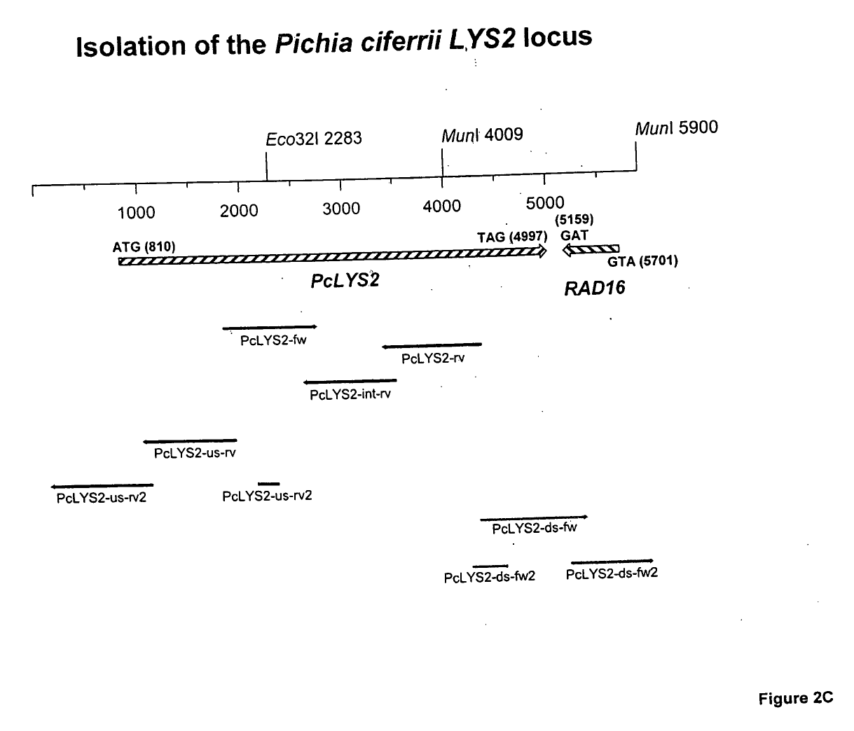

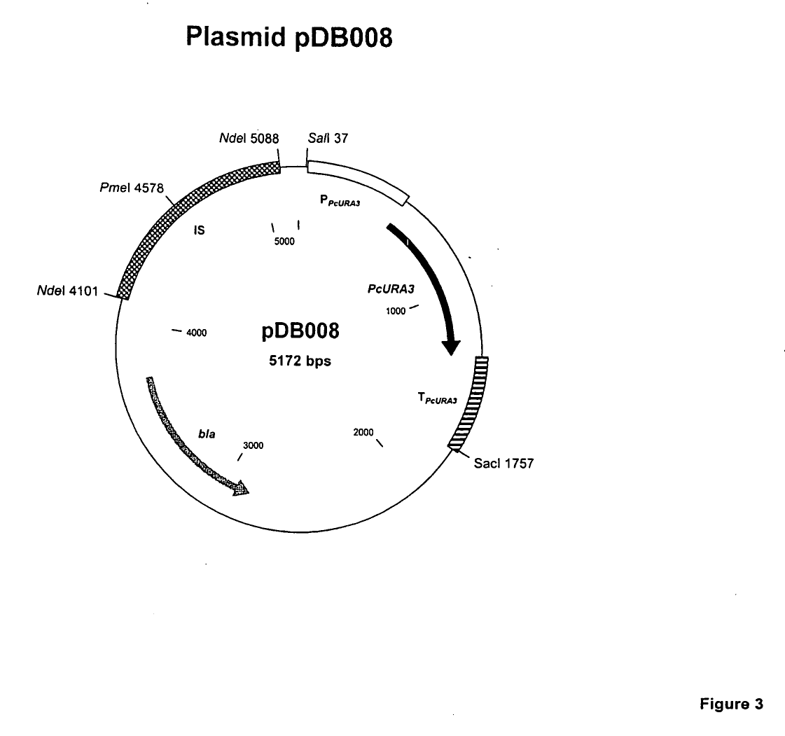

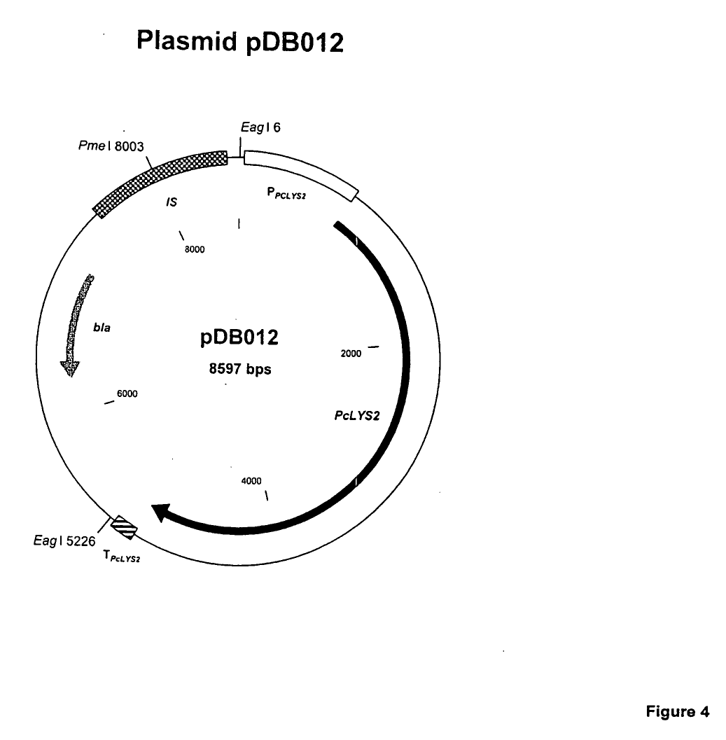

were used. DNA sequencing was performed by Sequiserve (Vaterstetten, Germany). The generated sequence information (2500 bp, corresponding to nt 1794-4293 in SEQ ID NO: 3; FIG. 2) was translated into protein using the Clone Manager 7 software (Scientific & Educational Software) and the resulting amino acid sequence used as template for a BLASTP search with NCBI's non-redundant protein database (www.ncbi.nlm.nih.gov/BLAST/). The search resulted in the identification of Candida albicans Lys2p (NCBI acc.# EAL02670), as being the protein in the database most similar to the new sequence, confirming that in fact portions of the Pichia ciferrii LYS2 ortholog had been amplified.

Amplification of the Entire Pichia ciferrii LYS2 Gene and Determination of its DNA Sequence

In order to determine the DNA sequence of the entire Pichia ciferrii LYS2 gene (coding sequence, promoter region and 3′-untranslated region) an inverse PCR approach was followed. Chromosomal DNA (300 ng) from Pichia ciferrii F-60-10A NRRL 1031 (isolated as described in Example 1) was digested overnight with Eco321 (MBI Fermentas, cat.# ER0301) according to the instructions of the manufacturer in a total volume of 50 μl. The digested DNA was purified using the QIAquick PCR Purification Kit (Qiagen, cat.# 28106) according to the instructions of the manufacturer. The eluted DNA (50 μl) was subjected to overnight ligation using the T4 DNA Ligase (New England Biolabs, cat.# M0202L) according to the instructions of the manufacturer in a total volume of 200 μl with 800 U of T4 DNA Ligase. The ligated DNA was purified using the QIAquick PCR Purification Kit according to the instructions of the manufacturer. 2.5 μl of the eluate was used as template for an inverse PCR reaction according to Innis et al., (PCR protocols. A guide to methods and applications, 1990, Academic Press). To that end two oligonucleotides hybridizing with the already known portion of the Pichia ciferrii LYS2 gene were applied:

| | |
|---|---|
| PcLYS2-us-fw: | |
| CAT CAA AGA TGA ATT AGA TGT TGT TTC | (nt 1910-1936 |
| | in |
| | SEQ ID NO: 3) |
| and | |
| PcLYS2-us-rv: | |
| CAA CAA GTT CAT CCA AGA CAC C. | (nt 1902-1881 |
| | in |
| | SEQ ID NO: 3) |

Amplification was performed with Phusion™ High Fidelity PCR Master Mix according to the instructions of the manufacturer. Using this procedure a 4.0 kbp PCR product could be obtained. The fragment was purified using the MinElute PCR Purification Kit (Qiagen, cat.# 28006) according to the instructions of the manufacturer. The DNA sequence of this fragment was determined as described previously, using oligonucleotides PcLYS2-us-rv and

| | |
|---|---|
| PcLYS2-us-rv2: | |
| CCT TCA ACT TTC GAT ATT ATA G | (nt 976-955 in |
| | SEQ ID NO: 3) |

as sequencing primer. The newly obtained sequence information covered nt 1-1793 in SEQ ID NO: 3. No new sequence information downstream of the already known DNA sequence could be obtained as the primers used for inverse PCR anneal upstream of the Eco32I site (FIG. 2). In order to obtain the DNA sequence of the 3′-end of the coding region of the Pichia ciferrii LYS2 gene and its 3′-untranslated region another round of inverse PCR had to be performed. Therefore, the above described experimental protocol was repeated, except that MunI (MBI Fermentas, cat.# ER0751) was used for digesting Pichia ciferrii chromosomal DNA and the following oligonucleotides, synthesized by MWG Biotech (Ebersberg, Germany), were employed during inverse PCR:

| | |
|---|---|
| PcLYS2-ds-fw: | |
| TAA AGA TGG TAT TCC AGA ATC TGA TG | (nt 4214-4239 |
| | in |
| | SEQ ID NO: 3) |
| and | |
| PcLYS2-ds-rv: | |
| AGA TAA TTC AAC GAA ATG TTG AGT ATC. | (nt 4193-4167 |
| | in |
| | SEQ ID NO: 3) |

Using this procedure a 1.9 kbp PCR product could be obtained. The fragment was purified using the MinElute PCR Purification Kit according to the instructions of the manufacturer. The DNA sequence of this fragment was determined as described previously with oligonucleotides PcLYS2-ds-fw and

| | |
|---|---|
| PcLYS2-ds-fw2: | |
| ACC TTT GTA CAT ATG AAA TCT C | (nt 5138-5159 in |
| | SEQ ID NO: 3) |

as sequencing primers. 1608 bp of new sequence information (nt 4294-5900 in SEQ ID NO: 3) could be obtained which stretches to the next MunI site (FIG. 2). Using the described three-step procedure, a total of 5900 bp of the Pichia ciferrii LYS2 locus could be isolated and its DNA sequence be determined (see SEQ ID NO: 3 and FIG. 2).

The Pichia ciferrii LYS2 locus as depicted in FIG. 2 encodes the PcLys2p protein of 1395 amino acids in length (SEQ ID NO: 4). PcLys2p has 65% (79%) and 61% (75%) positional amino acid identity (similarity) to the α-aminoadipate reductase from Candida albicans (GenBank acc.# EAL02670) and the hypothetical α-aminoadipate reductase from Kluyveromyces lactis (GenBank acc.# XP_{—}451939), respectively. The Candida albicans LYS2 gene has been shown to complement Saccharomyces cerevisiae lys2 mutations (Suvarna et al. Molecular analysis of the LYS2 gene of Candida albicans: homology to peptide antibiotic synthetases and the regulation of the α-aminoadipate reductase. Current Genetics, 33: 268-275, 1998). It has been characterized biochemically and been shown to have α-aminoadipate reductase activity (Guo et al. Novel posttranslational activation of the LYS2-encoded α-aminoadipate reductase for biosynthesis of lysine and site-directed mutational analysis of conserved amino acid residues in the activation domain of Candida albicans. Journal of Bacteriology, 183: 7120-7125, 2001). Additionally the PcLys2p contains a highly conserved phosphopantetheinylation domain (LGGHSI, amino acid residues 881 to 886 in SEQ ID NO: 4) (Suvarna et al. Molecular analysis of the LYS2 gene of Candida albicans: homology to peptide antibiotic synthetases and the regulation of the α-aminoadipate reductase. Current Genetics, 33: 268-275, 1998).

### Example 8

Construction of a Vector for Complementation of Lysine-Auxotrophic Pichia ciferrii Strains

In order to complement the lysine auxotrophy of the lysine auxotrophic Pichia ciferrii strain (see example 6) we constructed an integrative LYS2 expression vector.

To that end 500 ng of chromosomal DNA of Pichia ciferrii F-60-10A NRRL 1031 (isolated as described in Example 1) was used as template for a PCR according to Innis et al. (PCR protocols. A guide to methods and applications, 1990, Academic Press) to amplify the α-aminoadipate reductase gene (coding sequence including promoter and terminator) of Pichia ciferrii (LYS2). For this the following oligonucleotides were applied:

PcLYS2-compl-fw (nt 1-36 in SEQ ID NO: 3):
5′-AAT TAT TCT TAT CTT TAA CAT CAT TAC CAC TTA
ACC-3′
and
PcLYS2-compl-rv (nt 5158-5117 in SEQ ID NO: 3):
5′-AGA TTT CAT ATG TAC AAA GGT TAA TAC ACA TAT AAT
ATA TTC-3′.

The fragment was purified using the QIAquick PCR Purification Kit according to the instructions of the manufacturer. Ligation into vector pCR®-Blunt II-TOPO® and transformation of chemically competent Escherichia coli cells was performed according to the Zero Blunt® TOPO® PCR Cloning Kit (Invitrogen Corporation, Carlsbad). The orientation and authenticity of the insert was determined by DNA sequencing.

The resulting vector was digested with Eagl (New England Biolabs, cat.# R0505S) according to the instructions of the manufacturer, followed by gel extraction of the 5220 bp insert carrying the Pichia ciferrii LYS2 gene using QIAquick Gel Extraktion Kit (Qiagen, cat.# 28706) according to the instructions of the manufacturer and ligation of the 5220 bp insert into the backbone of vector pTH-GAP-nat1-IS2-PmeI (described in example 4) obtained by digestion with EagI (3377 bp), generating vector pDB012. The orientation and authenticity of the insert was determined by restriction analysis. Ligation, preparation and transformation of chemically competent Escherichia coli cells was performed by methods known to the skilled person.

### Example 9

Complementation of Lysine-Auxotrophic Pichia ciferrii Strains with Plasmid pDB012

To complement lysine-auxotrophic Pichia ciferrii cells, they were grown in YEPD medium to an OD₆₀₀ of 1 to 1.5. The cells were harvested by centrifugation and resuspended in 0.1 culture volume of 50 mM phosphate buffer (pH 7.5) to which 25 mM dithiothreitol was added. After incubation at 37° C. for 15 min, the cells were washed twice with a culture volume of cold stabilization solution (270 mM sucrose, 10 mM Tris-HCl [pH 7.5], 1 mM MgCl₂), and resuspended in 0.01 culture volume of stabilization solution. 2.5 μl vector pDB012 linearized by digestion with PmeI (see Example 8) (containing 1.0 μg DNA) or, as a negative control, 2.5 μl H₂0 were mixed with 50 μl of cell suspension and incubated on ice for 10 min. Then the transformation mixture was transferred to a 2 mm electroporation cuvette. Electroporation was performed with a GenePulser Xcell at 500 V, 50 μF and 700 Ω (Bio-Rad Laboratories, Hercules, U.S.A.). After electroporation the cells were resuspended in 500 μl of stabilization solution and transferred to a culture tube containing 2 ml YPD medium. After overnight regeneration at 30° C. and 250 rpm, aliquots of the regenerated cells were plated on YNB medium lacking lysine and incubated at 30° C. While no colonies appeared on plates containing the cells treated with water only, transformation of lysine-auxotrophic Pichia ciferrii cells with linearized vector pDB012 did result in formation of colonies on plates containing these cells after three days of incubation. Single colonies were analyzed for correct integration of plasmid pBD012 into the 5S-26S rDNA intergenic spacer via colony PCR. To that end the following oligonucleotides were synthesized by MWG Biotech (Ebersberg, Germany):

PcL41 -up:
GCG TTC TAA CGA CAA TAT GTC C
and
26S rDNA-down:
GTT TGT GTT TAT CAC AAC ACT TCT AAG.

These oligonucleotides were used to set up a PCR reaction according to Innis et al., (PCR protocols. A guide to methods and applications, 1990, Academic Press) with Taq PCR Master Mix (Qiagen, cat.# 201445) according to the instructions of the manufacturer and cells from lysine-prototrophic colonies as template. Only in case of correct integration of pDB012 in the 5S-26S rDNA intergenic spacer, the PCR reaction would yield a 1040 bp fragment, demonstrating that integration of vector pDB012 harbouring the Pichia ciferrii LYS2 gene did take place at the 5S-26S rDNA intergenic spacer of Pichia ciferrii. All colonies tested led to amplification of the 1040 bp fragment. Consequently, transformation of lysine-auxotrophic Pichia ciferrii cells with vector pDB012 containing the Pichia ciferrii LYS2 gene resulted in complementation of the biosynthetic defect.

1. A Pichia ciferrii cell that is defective in a biosynthetic pathway.

2. The Pichia ciferrii cell of claim 1 wherein the biosynthetic pathway is a pathway for a nucleotide, an amino acid and/or a vitamin.

3. The Pichia ciferrii cell of claim 1, wherein the biosynthetic pathway is a pathway for uracil and/or for lysine.

4. The Pichia ciferrii cell of claim 3, wherein the biosynthetic pathway for uracil is defective in the activity of the enzyme orotidine-5-phosphate decarboxylase.

5. A Pichia ciferrii cell of claim 3, wherein the biosynthetic pathway for lysine is defective in the activity of the enzyme a-aminoadipate reductase.

6. A Pichia ciferrii transformation system comprising the Pichia ciferrii cell of claim 1 as a host cell and a polynucleotide comprising a functional gene that complements the defect in the biosynthetic pathway in which the host cell is defective.

7. The Pichia ciferrii transformation system of claim 6 wherein the functional gene is the URA3 gene and/or the LYS2 gene.

8. A method for transformation of Pichia ciferrii comprising transforming the Pichia ciferrii cell of claim 1 with a polynucleotide comprising a functional gene that complements the defect in the biosynthetic pathway and isolating transformed cells that are prototrophic for the defect in the biosynthetic pathway.

9. The method according to claim 8 wherein the functional gene is the URA3 gene and/or the LYS2 gene.

10. A polynucleotide for use in a method according to claim 8 comprising a functional gene from Pichia ciferrii that complements the defect in said biosynthetic pathway in which the host cell is defective.

11. A polynucleotide for use in a method according to claim 8 comprising a functional gene that complements the defect in said biosynthetic pathway in which the host cell is defective, wherein the functional gene has a DNA sequence encoding a polypeptide with an amino acid sequence of SEQ ID NO: 2, an amino acid sequence of SEQ ID NO: 4, an amino acid sequence having a degree of identity of at least 91% to the amino acid sequence of SEQ ID NO: 2 or an amino acid sequence having a degree of identity of at least 70% to the amino acid sequence of SEQ ID NO: 4.

12. A method for the production of a compound of interest comprising transforming the Pichia ciferrii cell of claim 1 with a polynucleotide containing a functional gene complementing the defect in said biosynthetic pathway in which the host cell is defective and a polynucleotide comprising a DNA sequence of interest, selecting transformed cells for prototrophy, culturing transformed cells under conditions conducive to production of the compound of interest, and, optionally, recovering the compound of interest.

13. The method according to claim 12 wherein the functional gene is the URA3 gene and/or the LYS2 gene.

14. A polypeptide that complements a defect in the biosynthetic pathway for uracil or lysine of Pichia ciferrii and has an amino acid sequence of SEQ ID NO: 2 or SEQ ID NO: 4, respectively, or an amino acid sequence having a degree of identity of at least 91% to the amino acid sequence of SEQ ID NO: 2 or an amino acid sequence having a degree of identity of at least 70% to the amino acid sequence of SEQ ID NO: 4.


SEQUENCE LISTING (4 sequences)

SEQ ID NO: 1 - DNA, 2359 bp, Pichia ciferrii
  CDS (1072)..(1875): Product Orotidine-5′-phosphate decarboxylase
aacaatttaatactgaaataaaagttgtaatatagtataaaatataaaattcatgagaaatatgtacaaaaggtttgtaagatcaccacattttctttaaaatgatctcaccataataataaaacatctaaagataaatcaaagggaacccacaaattgaaatgaaaagaattgtataaacatcatgactttaaacaattcacttctagtatatgagagataataaattcattaacagtattttttttttctaaattttttaattttttkywtstamrkkmywyrakymwayrarwawawwwgwyyratrmryykrwtaaatatctgaaccttaacgtctcatttgttgagttaataaagcattttgttcttggaatctttgaccgactctttcactgtgtttcaagaatttttcagaacattttaaaacacatgtattttcttttgaagttaatgaagcggaagtgaaatcattaacacaatcattgaaacaacgttcaaccaagtttgagtaaagacgcatgaaatctttcatttgtttaccttcaacgattctttggaaatcttgttgttctctttggtttaattgatccattttgattgatttgatatagatttgagctttgtagttctgatcaattaggtacaaaaggtctttcaatggcaaaagtgtacttttttgatttttcaatgaagtcgaaaaggttcaaatttttttttggtcaacaaaacatcataaatatccgaatttggaaagattcccgatatggacaaaatcaggaatcaattggaattatgattcatttgagcattatagtgtgaataattctttatgttctctgttctttattatatcaagctttaaagagacactaggcgtcccatagatcggatggtctattcaagtgtctcaaagaaaaaaaatagttgcttgattttggctgtagtagagcttgtacgggacacgcttctatgaataaatttgtccgatgagttggataaatattaactgaactaatttcttctaattgatagagcatcatcaatatattgcacataatataattcaaaatggttaccacattatcttattctcaaagagcagaagctcacccttctccattggcaaagcgtttattcaatgttatggaatcaaaaaagtcaaacttgtgtgcttcaattgatgttagaactacaaaagaatttttacaattagttgacacattaggtccatatatctgtcttgtcaaaactcatatcgatattattgatgatttctcattcgaaggtaccattaaaccattgaaagaattagcacaaaagcacaattttttaatctttgaagatcgtaaatttgctgatatagggaacacagtcaaagcacaatatgcaggtggtgctttcaaaattgcaacttggtctgatatcacaaatgctcatggtgttactggtgctggtatcgttagtggattgaaagaagctgctaaagaagcttctgatgaaccaagagggttgttgatgttggctgaattgagttcaaaaggttcattagccaaaggtgaatatactaaacaaactgttgatatcgctaaaactgataaagatttcgttattggttttattgctcaaaatgatatgggtggtagagatggaggctttgattggttaattatgaccccaggtgttggtcttgatgataaaggtgatgccttaggtcaacaatatagatccgttgataccgttgtttcaactggtagtgatatcattattgttggtagaggtttgttcggtaaaggaagagatccaaatgctgaaggtgaacgttacagagctgctggttggaatgcctatttgaaaagaactggtcaaatatagtatgtagtttaaatctaacatattagactaaataattaatgggtcataatttgtaattagttcaacgactaactaactgacattagaaggaagactctaattgtataccatgtcatcaatactcctaattatcaaataaatcaaaatatataccttctatcattaattagaagatgtaatgtcgatgactaagcaagataaattaaaaccgaaatccatctgtatgtaaaaatactttcaagtaattaccacatagatagaagctcaccaaccaactatcatcaagccagtagatgccattcaagagtgacaaatgtcattggaaaatgagttcacaggatcctcctttgagaatgagtttgaaaatactactgcccaaccagaattatcaaataaaggatcgagattgattggtgctagtaccgcaggtataagagctctaatgtatcaattaaccactatatacttaagaacccctccagca

SEQ ID NO: 2 - protein, 267 aa, Pichia ciferrii
MVTTLSYSQRAEAHPSPLAKRLFNVMESKKSNLCASIDVRTTKEFLQLVDTLGPYICLVKTHIDIIDDFSFEGTIKPLKELAQKHNFLIFEDRKFADIGNTVKAQYAGGAFKIATWSDITNAHGVTGAGIVSGLKEAAKEASDEPRGLLMLAELSSKGSLAKGEYTKQTVDIAKTDKDFVIGFIAQNDMGGRDGGFDWLIMTPGVGLDDKGDALGQQYRSVDTVVSTGSDIIIVGRGLFGKGRDPNAEGERYRAAGWNAYLKRTGQI

SEQ ID NO: 3 - DNA, 5900 bp, Pichia ciferrii
  CDS (810)..(4997): Product alpha-aminoadipate reductase
aattattcttatctttaacatcattaccacttaaccaattctttatttgatctctttgtctttgtaatttcttgatttctcttttcaaatcaccttcaattttttccttttgagactgactttcacaattttgaagcttgtcatagtagccatcaaagatctctagaccttctttaactttcttgaagactttatcaatctcttgtaataattttctttgtgacattgttaaatctttgtttaaatctgtatatgatatgaaactgtgtgtttatcgaagcacttttctcactcaattggaacgttttggacttgtaagaaaaccagcggatagactattgaacacgtagaattcaaaaacaattaaaaaacagcacaattaccaactattcaacacagaattcaccgttgtttgatccaccaaaattgtcacgtctctattattccagcgcttttttctgattctttccactttttttggtgagcaaatcgagatccgaccatttttaaagatcggaactcccggttttctagagttttttttttgacagccgatttccgaccgattttttgagattatcagtcataatgtttttgatgaaagattttgattgtatttgatattgttaactaagtggtaagagttcaagcaaggttttatcttggtttaaagtgagtaaaagtggtattgtatcaggaattatcaccattgtgctttgtttcgaggatcattggtgtaaatttcatccatttcacaaggatctcttgtactaacatttccaattgatatagatacattttattcaaaaatgtcttcagatttacaacattgggctcaaatattgagtagtccaactctctcagtattacctcatgatttcattcgtccttctaacaacaaagttgttgaagctaaacacaatttttcaattccagcaaaagcattatcacaatctataatatcgaaagttgaaggttcaaatgaattcatcattggtctttcagtctttgcagttttgatctatagactaactggtgatgatgacattgttatctccaccgactctccaaatggcggtaaagaatttgtcattagattaaccattagtccaactgtttcatttgtggaattagttgaacaaattacaaaattatataatgacaatgtcaatagagctgttgattatgatgaattatctgcattgatccaaaaagaaaacggttatgaaactaaaccttctttattcagattaagtttccaatatggtaatgctcatcaacaattatctgaatatggttcaattagagatttatcaatctttgtcaatgatgattcttatgatgtctattttaatgctttattatacaaagatgatcgtatttcaattttggctgaacaatttaaccaatttttggaagctgtcgcccaagattcttcattacaagttactaaaataaatttaatcaccaaatctcaaaaacaaatcttaccagatccaacaactgatttagattggtctggttatagaggtgctatccatgatattttccaagaaaatgcagctaaattcccagaacgtacttgtgttgtggaaactccaagtttcttaacaccagaaaaacaagctagagtctttacttataaacatatcaatgaagcttcaaacattgttggtaactatttagttaaaactggtatcaaaagaggtgatgttgttatgatttattcttcaagaggtgttgatttaatggtttcagttctcggtgttttaaaagccggtgctactttctccgttattgatccagcttatcctccagctcgtcaaaatatttatttacaagttgctaaaccaaaaggtttaatcgtcatcaaaaaagctggtgtcttggatgaacttgttgaaaattacatcaaagatgaattagatgttgtttctcgtgttcctcaaattgaattacaagatgatggttcagtcctcggtggtaaaaacttggaaggtgaagatgtcttagctggtgtttcaacaagttcaactggtgttttggttggtccagattcaaacccaacattatctttcacatcaggttctgaaggtttaccaaaaggtgtcttgggtagacatttctctttagcttattatttcgattggatgtccaagacttttaaattaaactctgatgataaattcactatgctttcaggtattgcacatgatcctattcaaagagatatgtttacaccaatcttcttgggtgctcaattattagttccaactgctgatgatatcggtaccccaggtaaattagctgaatggatgggtaaatacggtgctactgttactcatttaactccagctatgggtcaattattatcagctcaagctacatctgaaattccatcattacatcatgctttctttgtaggtgatatcttgaccaaaagagattgtttaagattacaaacattagctaaaaatgttttcattgtcaatatgtatggtactactgaaactcaaagagctgtttcatattttgaagttgcttcaagagctcatgattcaacatttttagaatctcaaaaagatgtcatgccagctggtaaaggtatgttgaatgttcaattattagttgttaatagaaatgatacttctcaaacatgtggtgttggtgaagttggtgaaatttatgttcgtgctgctggtttagctgaaggttatcgtggattacctgaattaaatgaacaaaaattcgtcaaaaattggtatgttgatgctcaacaatggattgatcaagataatcaaaaaagtcaaaatgaaccatggagagaattttggaaaggtccaagagatagattatacagaactggtgatttaggtcgttatttaccagatggtaatactgaatgttgtggtagagctgatgatcaagtcaaaattcgtgggttccgtattgaattaggtgaaattgatacaaatatttctcaacatcctttagttcgtgagaatgttacattagttcgtaaagatgctaataatgaaccaactttaatttcttatattgttccaaaaactgaagttgctgatttagaaaatttcaaatcatcagttgaagaattagaaaataaagatgatcacgttgttagtggattagttggttatcgtttattaattaaagatatcaaggcatttttgaaaaaaagattagcttcatatgctatcccaacaattattgttccattccataaattaccattaaatccaaatggtaaagttgataaaccaaaattaccattcccaaccactgcacaactagaagctgttgctaaacattcaagtgccgaccaagatgattctcaattcagtgaaactgaaggtaaagttcgtgatgtttggttatcagtcttacctcaacgtccatcaactatttcaccagaagattcattctttgatttaggtggtcattcaatcttagctacaagaatgatttttgaattaagaaaacaattagttattgatttaccattaggtactatcttcaaattcccaaccattaaagcttttgctaaagaaatcgaacgtgttcaaaaaggtgatgatgtcgccttaccagctactagtggtgctgaaaatactgatgaggatttagcaaataaaccagctgaatattataaagatgctcaagaattaactgaaactgcattaaaatcaaaatatgcttcaagattaagtttctctaaaaatgaacaaatcagcatttttgtcaccggtgctactgggttcttaggttcattcattattgctgatttattgaaccgtaaatcaaacatcaaaatctttgctcatgttcgtgccgctgataaagttaaaggtctcgaaagattgaaaaaagctggtttaacctacggtaattggaaagatgaatttgcctcatcaattgaagttgttttaggtgatttatcaaaagaacaatttggtttagatttccaagcttggggtaacttgactgaaacaattgatgttattattcataatggtgctttagttcactgggtgtatccatatgataaattacgtgatgccaatgttatcggtactatcaacgttttgaatttagctgcttctggtaaggctaagtatttctcatttgtttcttcaacatcagctttagatactcaacatttcgttgaattatctgataaattactttctgaaggtaaagatggtattccagaatctgatgatttacaaggttctgcttacggtttaggttcaggttacggtcaaagtaaatgggctgctgaatatattatccgtcgtgctggtgaacgtggtttgaaaggtgctattattagaccaggttatgttactggtgaaactaatactggttcttcaaacactgatgatttcttattacgtatgttgaaaggttgtgctgaattaggtaaatatccaaacattaataacaccatcaatatggtccctgttgatcatgttgcaagagttgttgttgcagctgccttacatccaccaaaatcagaagaattagctgttgttcaagttactggtcatccacgtatcacatttattcaatttgttggtgcattgaaaaaatatggttatgaaattgaattagaagattatgttacttggaaatcatcattagaaaaatttgttttggaaggtactcaagaaagtgcactttatccattattacattttgttttagatgatttaccacaagatactaaagctccagaattagatgatgttaatgctcgtacttcattgaaagcagatgctgaatggactggcgttgatgcgtcagctggtagaggtattgatgaacaacaattaagcgtttacgttgcatacttgattaaagttggtttcttaccaaaaccatcaaaagctggcgaaccattaccagaagttaagttaactaaagaacaactcgatttgattacatctggtgctggttctcgtggtagttcaaattagatagatttgaagtaaaaaaaaaacataaattacgtctgtacatatagttaaaatattcgttaaataaattgatttatagattttgttacaattctgtagagtttgttatttattaaaccgaatatattatatgtgtattaacctttgtacatatgaaatctctaattcataaataaaaactgtaaatcatcaggggttaatctattaattgcaccatcatcatggttaatggtagcatgaatcatatttgccttcttatcttgtaattctataattcttgattctatactatcttcaatacaaaatcttgtaattttaacaggtctaaattgaccaattctatgaactctatcaccactttgccattcaacagatggattccaccaaggatccattaagaaaacttgtgaagcttcacataaatttaaagcaacaccaccagctttcaaactgaccaagaaaacttcaacatttgtattttccataaaatgacgaattgtattatctctttgaataggtgacattgaaccctgtaatttaacagtttgaaaacctgctcttttcaatctccattcaattaaatctaacatacttgtaaattgtgaaaacacaatagatttaatagtttgtctatcacttcttaatttataaagttcttctactaaagcttcaatctttgtagatgacctccattcaccacccattttaattctattaacaattgatgttattattcataatggtgctttagttcactgggtgtatccatatgataaattacgtgatgccaatgttatcggtactatcaacgttttgaatttagctgcttctggtaaggctaagtatttctcatttgtttcttcaacatcagctttagatactcaacatttcgttgaattatct

SEQ ID NO: 4 - protein, 1395 aa, Pichia ciferrii
MSSDLQHWAQILSSPTLSVLPHDFIRPSNNKVVEAKHNFSIPAKALSQSIISKVEGSNEFIIGLSVFAVLIYRLTGDDDIVISTDSPNGGKEFVIRLTISPTVSFVELVEQITKLYNDNVNRAVDYDELSALIQKENGYETKPSLFRLSFQYGNAHQQLSEYGSIRDLSIFVNDDSYDVYFNALLYKDDRISILAEQFNQFLEAVAQDSSLQVTKINLITKSQKQILPDPTTDLDWSGYRGAIHDIFQENAAKFPERTCVVETPSFLTPEKQARVFTYKHINEASNIVGNYLVKTGIKRGDVVMIYSSRGVDLMVSVLGVLKAGATFSVIDPAYPPARQNIYLQVAKPKGLIVIKKAGVLDELVENYIKDELDVVSRVPQIELQDDGSVLGGKNLEGEDVLAGVSTSSTGVLVGPDSNPTLSFTSGSEGLPKGVLGRHFSLAYYFDWMSKTFKLNSDDKFTMLSGIAHDPIQRDMFTPIFLGAQLLVPTADDIGTPGKLAEWMGKYGATVTHLTPAMGQLLSAQATSEIPSLHHAFFVGDILTKRDCLRLQTLAKNVFIVNMYGTTETQRAVSYFEVASRAHDSTFLESQKDVMPAGKGMLNVQLLVVNRNDTSQTCGVGEVGEIYVRAAGLAEGYRGLPELNEQKFVKNWYVDAQQWIDQDNQKSQNEPWREFWKGPRDRLYRTGDLGRYLPDGNTECCGRADDQVKIRGFRIELGEIDTNISQHPLVRENVTLVRKDANNEPTLISYIVPKTEVADLENFKSSVEELENKDDHVVSGLVGYRLLIKDIKAFLKKRLASYAIPTIIVPFHKLPLNPNGKVDKPKLPFPTTAQLEAVAKHSSADQDDSQFSETEGKVRDVWLSVLPQRPSTISPEDSFFDLGGHSILATRMIFELRKQLVIDLPLGTIFKFPTIKAFAKEIERVQKGDDVALPATSGAENTDEDLANKPAEYYKDAQELTETALKSKYASRLSFSKNEQISIFVTGATGFLGSFIIADLLNRKSNIKIFAHVRAADKVKGLERLKKAGLTYGNWKDEFASSIEVVLGDLSKEQFGLDFQAWGNLTETIDVIIHNGALVHWVYPYDKLRDANVIGTINVLNLAASGKAKYFSFVSSTSALDTQHFVELSDKLLSEGKDGIPESDDLQGSAYGLGSGYGQSKWAAEYIIRRAGERGLKGAIIRPGYVTGETNTGSSNTDDFLLRMLKGCAELGKYPNINNTINMVPVDHVARVVVAAALHPPKSEELAVVQVTGHPRITFIQFVGALKKYGYEIELEDYVTWKSSLEKFVLEGTQESALYPLLHFVLDDLPQDTKAPELDDVNARTSLKADAEWTGVDASAGRGIDEQQLSVYVAYLIKVGFLPKPSKAGEPLPEVKLTKEQLDLITSGAGSRGSSN